(19) United States
(12) Patent Application Publication
(10) Pub. No.: US 2011/0319485 A1
(43) Pub. Date: Dec. 29, 2011

(54) Composition Having Anti-Helicobacter Pylori Activity And Its Application To Inhibit Helicobacter Pylori

(75) Inventors: Hsiu-Man Lien, Taichung (TW); Chin-Jui Tseng, Taichung (TW); Ya-Yun Lai, Taichung (TW); Chao-Lu Huang, Taichung (TW); Chia-Chang Chen, Taichung (TW)

(73) Assignee: Yushen Biotechnology & Medical Co, Ltd., Taichung (TW)

(21) Appl. No.: 13/097,148

(22) Filed: Apr. 29, 2011

(30) Foreign Application Priority Data
Jun. 24, 2010 (TW) 099120578

(51) Int. Cl. A61K 31/365; A61P 31/04; A61P 1/04; C07D 493/18

(52) U.S. Cl. 514/468; 549/268

(57) ABSTRACT

The present invention provides a composition having anti-Helicobacter pylori activity and the composition contains ovatodiolide with effective dosage to inhibit Helicobacter pylori growth or ovatodiolide related substances and its acceptable carrier. The present invention also provides a method for treating ovatodiolide to produce a composition for killing Helicobacter pylori. The present invention also provides an application of ovatodiolide to produce pharmaceuticals for curing gastric ulcers. The ovatodiolide isolated from natural material extract has been confirmed able to inhibit the growth of Helicobacter pylori or to kill Helicobacter pylori. The ovatodiolide can be used in various industries related to the inhibiting or killing of Helicobacter pylori with few side effects and is easy to obtain with low cost.

### BACKGROUND OF THE INVENTION

1. Field of the Invention

The present invention relates to a composition that can be used as a Helicobacter pylori (H. pylori) inhibitor. The present invention also relates to an application about using ovatodiolide to produce a composition that can be used as a Helicobacter pylori inhibitor. The present invention also relates to an application to produce medical and pharmaceutical products containing ovatodiolide to treat gastric ulcer.

2. Description of the Prior Arts

Helicobacter pylori is a gram-negative, microaerophilic bacterium that can inhibit the human stomach and duodenum. Helicobacter pylori has been confirmed related to peptic ulcers and stomach cancer since being discovered in 1982. It causes chronic low-level inflammation of stomach lining, even gastric ulcers, duodenum ulcers and stomach cancer. Over 80% of individuals infected with H. pylori are asymptomatic.

The treatment of western medicine to cure H. pylori in the present time is combining various dosages of the following group: (1) antibiotics, e.g., amoxicillin, clarithromycin, tetracycline, metronidazole, etc; (2) bismuth, antacids or proton pump inhibitor, etc. Using one single kind of antibiotic causes H. pylori to develop drug resistance and even turn stronger. Thus, the treatment combines two kinds of antibiotics plus bismuth or proton pump inhibitor and the treatment is called “triple therapy”. The recommended course of treatment is mostly two weeks. The side effects during the course, such as nausea, diarrhea, flatulence and dizziness, make the patients less submissive of taking pills, which results in increased relapse rate of ulcers because of failure to kill all H. pylori in one time. H. pylori not being killed may further develop drug resistance to some antibiotics and become even harder to be eradicated.

Herbal materials in Taiwan are plenty in species with various curative effects. Some of the medicinal plants have anti-H. pylori activity. Compared to western medicine, herbal materials have fewer side effects and the patients have higher submissiveness of taking herbal material because they are natural. The prior studies reveal that Plumbago zeylanica L., Paederia foetida L., Anisomeles indica L., Impatiens balsamin L., Alpinia zerumbet., and Bonbax ceiba L. have high anti-H. pylori activity wherein the extract of Plumbago zeylanica L. has minimum inhibitory concentrations ranging from 0.32 to 1.28 mg/mL(Huang, Studies of anti-Helicobacter pylori activity of Plumbago zeylanica Linn., a Taiwanese folk medicinal plant, master thesis of National Chung Hsing University, 2003), and the extracts of Impatiens balsamin L. have minimum inhibitory concentrations ranging from 0.16 to 5.12 mg/mL (Cheng, Studies of anti-Helicobacter pylori activity of Impatiens balsamin L., a Taiwanese folk medicinal plant, master thesis of National Chung Hsing University, 2005). Compared to known antibiotics, the minimum inhibitory concentrations of metronidazole is 64 μg/mL, the minimum inhibitory concentration of clarithromycin is 0.25 μg/mL and the minimum inhibitory concentration of tetracycline is 1 μg/mL (A Vasquez et al., 1996, Journal of Microbiology, 34 (5): 1232-1234). The aforementioned data may reveal a slight difference according to the procedure of experiment, but they still can demonstrate the anti-Helicobacter pylori activity of the medicinal plants is weaker than the anti-Helicobacter pylori activity of the antibiotics.

To overcome the shortcomings, the present invention provides a composition which has anti-Helicobacter pylori activity and no side effects to obviate the aforementioned problems.

### SUMMARY OF THE INVENTION

To prevent the defects described aforementioned, the purpose of the present invention is to provide a compound or a composition which can inhibit Helicobacter pylori effectively. Compared to the antibiotics of prior technology, the present invention has lower minimum inhibitory concentration, lower minimum bactericidal concentration, and fewer side effects. While used on the individuals, it will not cause physical harm to the individuals and can be used in curing gastric ulcers caused by Helicobacter pylori.

In order to achieve said purpose, the present invention provides a composition having anti-Helicobacter pylori activity containing ovatodiolide with effective dosage to inhibit Helicobacter pylori growth or ovatodiolide related substances and its acceptable carrier.

According to the present invention, the ovatodiolide has the following chemical formula:

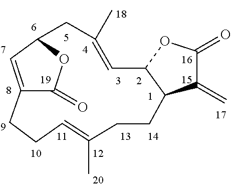

According to the present invention, the ovatodiolide related substances can be ovatodiolic acid, isoovatodiolide, 4-5-epoxovatodiolide, 4-hydroxy-5-enovatodiolide, 4-hydroxy-5-enovatodiolide, 4-methylene-5-hydroxyovatodiolide, 4-methylene-5-hydroxyovatodiolide, 4-methylene-5-oxovatodiolide whose anti-Helicobacter pylori activity is not being affected.

According to the present invention, the phrase “effective dosage to inhibit Helicobacter pylori growth” means the dosage has the ability to slow down, stop the growth of, or even kill the Helicobacter pylori. As the embodiments of the present invention, the minimum inhibitory concentration and the minimum bactericidal concentration can be detected in vitro.

The composition of the present invention can be taken orally or through a device being implanted in a body; preferably, the composition is taken orally. The composition of the present invention can be any known non-toxic, acceptable carrier, adjuvant or excipient for inhibiting the Helicobacter pylori growth.

Said composition can be any injection form sterilized, for example, sterilized, injectable aqueous suspension. The suspension can be made according to the prior art using suitable dispersion, lubricant and suspension. The acceptable excipient and solution include mannitol, water and isotonic sodium chloride solution.

The composition of the present invention can be taken orally through any orally acceptable form including, but not limited to, capsules, tablets, aqueous suspensions and aqueous solutions. As to tablets, the common carriers are lactose and corn flour. As to lubricant, the lubricant can be magnesium stearate. As to capsules, the usable dilution includes lactose and corn flour. As to aqueous suspension, the active compound, ovatodiolide, of the composition can be combined with emulsifier or suspension. As needed, the composition can be added with specific sweeteners and/or flavor enhancers and/or food colors.

In one aspect, the present invention also provides a method for killing Helicobacter pylori with ovatodiolide, which comprises administering Helicobacter pylori a dosage of ovatodiolide, such that growth of Helicobacter pylori is inhibited.

In another aspect, the present invention also provides a method for treating gastric ulcers with ovatodiolide, which comprises administering a therapeutically effective amount of ovatodiolide to a subject in need.

The term “a therapeutically effective amount”, used hereby, refers to kill Helicobacter pylori or to inhibit the growth of Helicobacter pylori.

The composition having anti-Helicobacter pylori activity of the present invention has been confirmed having the effects to inhibit the growth of Helicobacter pylori and compared to the prior art, the composition has lower minimum inhibitory concentration. Thus, the composition can be used as a new anti-Helicobacter pylori pharmaceutical to cure gastric ulcers caused by Helicobacter pylori. Because ovatodiolide is easy to obtain, isolate and purify by the method of the present invention and has few side effects, the ovatodiolide can be applied to various industries, such as health food, drinks and daily supplies.

Other objectives, advantages and novel features of the invention will become more apparent from the following detailed description when taken in conjunction with the accompanying drawings.

### BRIEF DESCRIPTION OF THE DRAWINGS

None.

### DETAILED DESCRIPTION OF THE PREFERRED EMBODIMENTS

The following embodiments provide various applications of ovatodiolide including a composition having anti-Helicobacter pylori activity and the use of ovatodiolide.

In an embodiment of the present invention, said composition having anti-Helicobacter pylori activity contains ovatodiolide or ovatodiolide related substances with effective dosage to inhibit Helicobacter pylori growth and its acceptable carrier. In an embodiment of the present invention, the acceptable carrier is aqueous solution.

In another embodiment of the present invention, the effective dosage to inhibit Helicobacter pylori growth is between 1 μg/mL and 5000 μg/mL.

In a preferred embodiment of the present invention, the effective dosage to inhibit Helicobacter pylori growth is between 5 μg/mL and 4000 μg/mL.

In another preferred embodiment of the present invention, the effective dosage to inhibit Helicobacter pylori growth is between 50 μg/mL and 3000 μg/mL.

According to the following embodiments, the ovatodiolide with the concentration ranging from 10 μg/mL to 20 μg/mL has the best effect of inhibiting Helicobacter pylori growth.

According to the present invention, the composition can be made into, for example, but not be limited to, food, cleaning supplies, and drinks. Such embodiments specifically include food additives, toothpaste and mouthwash.

In a preferred embodiment of the present invention, as to said method for treating ovatodiolide to produce a composition for killing Helicobacter pylori, the dosage to kill Helicobacter pylori is between 1 μg/mL and 5000 μg/mL.

In another preferred embodiment of the present invention, the dosage to kill Helicobacter pylori is between 5 μg/mL and 4000 μg/mL.

In yet another preferred embodiment of the present invention, the dosage to kill Helicobacter pylori is between 50 μg/mL and 3000 μg/mL. In an embodiment of the present invention, the dosage to kill Helicobacter pylori is 80 μg/mL.

The present invention was further illustrated by the following examples, but it should be understood that the examples and embodiments described herein are for illustrative purposes only, and should not be construed as limiting the embodiments set forth herein.

### Examples 1

### Chemicals and Reagents

Tryptic soy broth (TSB) and tryptic soy agar (TSA) were from Merck Co. (U.S.A). Brucella broth was from BD Co. (U.S.A). Dimethyl sulfoxide (DMSO), n-Hexane, ethyl acetate (EtOAc), acetonitrile (CH₃CN) and trifluoroacetic acid (TFA) were from Merck Co. (U.S.A). Clarithromycin (CLA) was from Zhejiang Guobang Pharmaceutical Co. Metronidazole (MTZ) was from FDC (U.S.A). The isolates and fraction solutions were further sterilized by passing through a 0.22 μm pore size filter (Millipore). The filtrate was collected in a sterile vial and stored at 4° C. until used.

2. Plant Material

Whole plants of Anisomeles indica (L.) Kuntze were collected in September 2005 from Yuli, Hualien County in Eastern Taiwan, and a botanically identified voucher specimen (YMT-05-03) was deposited in the Herbarium of the Institute of Biotechnology, Chaoyang University of Technology, Taiwan.

3. Plant Extract, Isolation and Purification of Ovatodiolide

The air-dried leaves (3.0 kg) of Anisomeles indica L. were extracted with deionic H₂O (80 L×2, 2 hrs per time) under reflux. After exhaustive extraction, the combined extracts were concentrated under reduced pressure to form dark brown syrup of approximately 119 g. The crude extract was then suspended in deionic H₂O and partitioned with hexane successively. The concentrated hexane layer (870 mg) was chromatographed on a silica gel column by eluting with a hexane/EtOAc gradient with increasing polarity, and yielded five fractions (Fr1-Fr5) were weighed 153, 78.7, 113, 291.3 and 130 mg, respectively. Fr4 was further separated by semi-preparative RP-HPLC (CH₃CN-0.1% TFA in H₂O, 63:37, UV detection at 265 nm), and yielded pure ovatodiolide (16 mg) (Shahidul Alam et al., 2000, Fitoterapia, 71: 574-576).

The HPLC system consisted of a Shimadzu LC-10AT HPLC pump, semi-preparative RP-HPLC (mightysil RP-18 GP 250×46 mm, i.d., 5 μm) equipped with a SIL-10AD auto-injector, an SPD-M10A diode array detector and a CTO-10A column oven. The mobile phase was CH₃CN-0.1% TFA in H₂O (63:37). UV detection wavelength was set at 265 nm. Data handling was performed by a Sigma-Plot software program.

4. Helicobacter pylori Strain and Growth Conditions

H. pylori BCRC 17027 was obtained from the Bioresources Collection and Research Center (BCRC), Hsinchu, Taiwan, ROC. The H. pylori strain was cultured in 5 mL tryptic soy broth (TSB, each liter containing: 17 g peptone from casein, 3 g peptone from soymeal, 2.5 g dextrose, 5 g sodium chloride, 2.5 g dipotassium phosphate, pH 7.3) with a tryptic soy agar (TSA, each liter containing: 15 g peptone from casein, 5 g peptone from soymeal, 5 g sodium chloride, 15 g agar, pH 7.3) and slant containing 5% (v/v) of defibrinated sheep blood formed at the bottom of the test tube.

The broth was incubated in a microaerophilic jar system (BBL), featuring a gas composition of 5% O₂, 10% CO₂ and 85% N₂ (BD GasPak™ EZ Gas Generating systems), at 37° C. for 72 hours. The cell suspension was then diluted with TSB to provide a cell concentration of 3×10⁸ CFUs/mL for antimicrobial testing.

5. The Anti-Helicobacter pylori Activity

(1) Inhibitory-Zone Measurement

An agar well diffusion assay was used to determine if H. pylori was susceptible to the anti-bacterial activity of the various fractions and isolates (al Somal et al., 1994, J R Soc Med, 87: 9-12). A volume of 100 μL for each of the bacterial suspensions tested (3×10⁸ CFUs/mL) was spread onto the TSA agar plate containing 5% (v/v) defibrinated sheep blood. Wells sized 7 mm in diameter were punched on the plates with 5 μL of the Fr1-Fr5 and ovatodiolide (40 mg/mL; DMSO as solvent) to be individually incorporated into the wells. DMSO was used as negative control and metronidazole (MTZ, 40 mg/mL) was used as positive control. Diameters of inhibition zones were determined after 72 hours of incubation at 37° C. under microaerophilic jar system, featuring a gas composition of 5% O₂, 10% CO₂ and 85% N₂.

(2) Minimum Inhibitory Concentrations (MIC) Determination.

The MIC of the purified substance was determined by the agar-dilution method (Shungu et al., 1987, Antimicrob Agents Chemother, 31: 949-950). A volume of 40 μL of cell suspension (initial bacterial count 3×10⁸ CFUs/mL) was spread onto the TSA agar plates containing 5% (v/v) defibrinated sheep blood. Final concentrations of ovatodiolide and the various fractions in the medium were set to be 2.0-32.0 μg/mL and 32.0-512.0 μg/mL with DMSO concentration lower than 2.5%, respectively. DMSO was used as control. Metronidazole (MTZ) and clarithromycin (CLA) were co-assayed as the positive reference at concentrations of 8.0-128.0 μg/mL and 0.008-0.128 μg/mL, respectively. Following incubation in a microaerophilic jar system, featuring a gas composition of 5% O₂, 10% CO₂ and 85% N₂, at 37° C. for 72 hours, the colonies that had formed on the plates were enumerated. The MIC was defined as the minimum concentration of the test sample (antibacterial agent) in a given culture medium on which bacteria are not able to form colonies.

(3) Minimum Bactericidal Concentration (MBC) Determination

Broth dilution MBC was determined in cryogenic vials using two-fold serial dilutions of isolates and control antimicrobial agents (Clarithromycin, metronidazole) (Zaidi et al., 2009, Biol Pharm Bull, 32: 631-636). H. pylori were suspended in Brucella broth and diluted to reach at a cell concentration of 1×10⁷ CFUs/mL). Final concentrations of each ovatodiolide were 10-160 μg/mL, and for the various fractions were 80-1280 μg/mL, and for the Clarithromycin were 0.25-4 μg/mL, and for the metroniazole were 160-2560 μg/mL in the mixture. The mixture was incubated in a microaerophilic condition at 37° C. for 1 hour. Following incubation, 100 μL aliquots of the broth were plated onto TSA plates containing 5% (v/v) sheep blood to determine the viable CFUs. The plates were incubated in a microaerophilic jar system, featuring a gas composition of 5% O₂, 10% CO₂ and 85% N₂, at 37° C. for 72 hours. The MBC was defined as the lowest concentration of the tested sample to completely inhibit visible bacterial growth on TSA plate. The final DMSO concentration in the assay never exceeded 2% (v/v) and did not have any effect on the growth at this concentration.

6. Statistical Analysis

All data are expressed as mean±S.D. and accompanied by six distinct experiments. Statistical analysis was performed using Dunnett's one-way ANOVA test, and the significant difference was set at p value<0.001.

### EXPERIMENT RESULTS

1. Inhibitory-Zone Measurement

Table 1 displays the results for the in vitro evaluation of anti-Helicobacter pylori activity of the various fractions and of the ovatodiolide from Anisomeles indica stem. Among the stem extracts and fractions, the ones that showed activity were Fr1, Fr2, Fr3, Fr4 and Fr5, Fr4 being the most active (33 mm inhibition halo). The ovatodiolide showed the greatest activity and potency among the tested products, with inhibition halos of 34 mm at 200 μg/disk. Metronidazole, used as the standard drug in the assay, presented inhibition halos smaller than ovatodiolide.

TABLE 1
Anti-H. pylori activity spectra of five fractions from water
extract partitioned with hexane and chromatographed on a
silica gel column by eluting with a hexane/EtOAc gradient.

| Fractions | The diameter of inhibitory-zone (mm)^{a} |
|---|---|
| Fr1 | 18.1 ± 2.8 |
| Fr2 | 20.5 ± 1.8 |
| Fr3 | 27.9 ± 3.2 |
| Fr4 | 33.0 ± 2.2 |
| Fr5 | 22.1 ± 3.1 |
| Hexane layer | 26.5 ± 1.8 |
| Aqueous layer | — ^{b} |
| Ovatodiolide | 34.4 ± 2.0 |
| Metronidazole ^{c} | 21.8 ± 3.1 |

^{a}Disk size: 7 mm (diameter)
^{b} Absence of inhibitory zone
^{c} Reference antibiotics

2. Minimum Inhibitory Concentrations (MIC)

The n-hexane layer was fractionated by using silica gel column chromatography and ovatodiolide was isolated from the layer. The antibacterial activities of these factions are shown in Table 2. Anti-H. pylori activity of ovatodiolide isolated from Fr4 was tested in vitro and was measured as minimum inhibition concentration (MIC) values by using agar dilution method. Positive controls showed positive results as microorganism growth whereas negative controls did not show microbial growth as an indication of sterility of media and test condition. The MICs of the reference antibiotics (MTZ and CLA) were 64 and 0.032 μg/mL, respectively. No antimicrobial activity was determined with pure DMSO as control. The ovatodiolide exhibited the highest inhibitory effect on Helicobacter pylori at the concentration of 16 μg/mL

TABLE 2
In vitro minimum inhibitory concentration(MIC) (μg/mL)
and minimum bactericidal concentration (MBC) (μg/mL)
of ovatodiolide against Helicobacter pylori BCRC
17027 strain

| | Agar dilution assay | | Broth dilution assay | |
|---|---|---|---|---|
| | MIC (μg/mL) | | MBC^{a} (μg/mL) | |
| Fractions | MIC range | MIC | MBC range | MBC |
| Fr1 | | >512 | | >1280 |
| Fr2 | | >512 | | >1280 |
| Fr3 | 32-512 | >512 | 80-1280 | >1280 |
| Fr4 | | 256 | | 320 |
| Fr5 | | >512 | | 320 |
| Ovatodiolide | 2-32 | 16 | 10-160 | 80^{b} |
| Clarithromycin^{c} | 0.008-0.128 | 0.032 | 0.25-4 | 1 |
| Metronidazole^{c} | 8-128 | 64 | 160-2560 | 2560 |

^{a}Values are means of three experiments.
^{b}The significant difference was set at p < 0.05
^{c}Reference antibiotics

3. Minimum Bactericidal Concentration (MBC)

The MBC of pure isolates was determined against H. pylori. The MBC ranged from 10 to 160 μg/mL for pure isolates. The lowest MBC was 80 μg/mL recorded for the ovatodiolide (Table 2). The MBCs of standard drugs clarithromycin and metronidazole that served as the positive control were 1 and 2560 μg/mL, respectively. The MBC values were found to be lower than the anti-bacterial values tested, suggesting that the isolate was bacteriostatic at lower concentrations and bactericidal at higher concentrations.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A composition having anti-Helicobacter pylori activity containing ovatodiolide with effective dosage to inhibit Helicobacter pylori growth or ovatodiolide related substances and its acceptable carrier.

2. The composition having anti-Helicobacter pylori activity according to claim 1, wherein the effective dosage to inhibit Helicobacter pylori growth is between 1 μg/mL and 5000 μg/mL.

3. The composition having anti-Helicobacter pylori activity according to claim 1, wherein the effective dosage to inhibit Helicobacter pylori growth is between 5 μg/mL and 4000 μg/mL.

4. The composition having anti-Helicobacter pylori activity according to claim 1, wherein the effective dosage to inhibit Helicobacter pylori growth is between 50 μg/mL and 3000 μg/mL.

5. The composition having anti-Helicobacter pylori activity according to claim 1, wherein the acceptable carrier is aqueous solution.

6. The composition having anti-Helicobacter pylori activity according to claim 2, wherein the acceptable carrier is aqueous solution.

7. The composition having anti-Helicobacter pylori activity according to claim 3, wherein the acceptable carrier is aqueous solution.

8. The composition having anti-Helicobacter pylori activity according to claim 4, wherein the acceptable carrier is aqueous solution.

9. A method for killing Helicobacter pylori with ovatodiolide, which comprising administering Helicobacter pylori a dosage of ovatodiolide.

10. The method for killing Helicobacter pylori with ovatodiolide, according to claim 10, wherein the dosage to kill Helicobacter pylori is between 1 μg/mL and 5000 μg/mL.

11. The method for killing Helicobacter pylori with ovatodiolide, according to claim 10, wherein the dosage to kill Helicobacter pylori is between 5 μg/mL and 4000 μg/mL.

12. The method for killing Helicobacter pylori with ovatodiolide according to claim 10, wherein the dosage to kill Helicobacter pylori is between 50 μg/mL and 3000 μg/mL.

13. An application of ovatodiolide used to produce pharmaceuticals for curing gastric ulcers.
